(19) United States
(12) Patent Application Publication
(10) Pub. No.: US 2011/0263719 A1
(43) Pub. Date: Oct. 27, 2011

(54) POLYMORPHIC FORM OF RASAGILINE MESYLATE

(76) Inventors: Vinayak Gore, Maharashtra (IN); Bindu Manojkumar, Maharashtra (IN); Sandeep Sonawane, Maharashtra (IN); Dattatrey Kokane, Maharashtra (IN); Sinderpal Tank, Maharashtra (IN)

(21) Appl. No.: 13/056,012

(22) PCT Filed: Jul. 29, 2009
(86) PCT No.: PCT/GB09/50941

§ 371 Date: Jul. 12, 2011

(30) Foreign Application Priority Data
Jul. 30, 2008 (IN) 1289/KOL/2008

(51) Int. Cl. A61K 31/135; A61P 25/16; A61P 25/18; A61P 25/24; A61P 25/00; C07C 211/42; A61P 25/28

(52) U.S. Cl. 514/657; 564/428

(57) ABSTRACT

The present invention relates to a novel crystalline form of rasagiline mesylate and a pure form of rasagiline mesylate and processes for their preparation. Further, the invention relates to pharmaceutical compositions comprising said forms and use of said compositions in the treatment of patients suffering from Parkinson's Disease, dementia, Alzheimer's Disease, depression, hyperactive syndrome, stroke, brain ischemia, neurotrauma, schizophrenia and multiple sclerosis.

### FIELD OF THE INVENTION

The present invention relates to a novel crystalline form of rasagiline mesylate and a pure form of rasagiline mesylate and processes for their preparation. Further, the invention relates to pharmaceutical compositions comprising said forms and use of said compositions in the treatment of patients suffering from Parkinson's Disease, dementia, Alzheimer's Disease, depression, hyperactive syndrome, stroke, brain ischemia, neurotrauma, schizophrenia and multiple sclerosis.

### BACKGROUND OF THE INVENTION

Rasagiline, represented by structural formula (1) and chemically named 1-(R)-(2-propynylamino)indan, is a selective and potent irreversible monoamine oxidase type B (MAO-B) inhibitor. It is currently marketed, as the mesylate salt, for the treatment of Parkinson's Disease, both as monotherapy and as adjunct therapy to levodopa. Rasagiline may also be useful for the treatment of dementia, Alzheimer's Disease, depression, hyperactive syndrome, stroke, brain ischemia, neurotrauma, schizophrenia and multiple sclerosis.

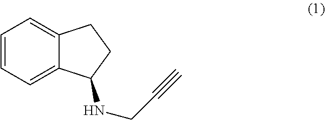

The manufacturing process for many pharmaceuticals is hindered by the fact that the compound which is the active pharmaceutical ingredient (API) is amorphous or has an irregular or unstable crystalline form. In some cases, such irregularities can cause handling difficulties during the manufacturing process and/or undesirable properties being imparted to the final drug or dosage form. The latter include inconsistent drug substance dissolution rates and bioavailability.

Polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different X-ray diffraction peaks. Polymorphic forms of a compound can be distinguished in a laboratory by X-ray diffraction spectroscopy and by other methods such as infrared spectrometry. Additionally, the properties of polymorphic forms of the same active pharmaceutical ingredient are well known in the pharmaceutical art to have an effect on the manufacture of drug product compositions comprising the API. For example, the solubility, stability, flowability, tractability and compressibility of the API as well as the safety and efficacy of the drug product can be dependent on the crystalline from.

Rasagiline was first described in U.S. Pat. No. 5,457,133, in which the method of its preparation is a single step process comprising reacting (R)-1-aminoindan with propargyl chloride. The reaction is carried out in acetonitrile with potassium carbonate at a temperature of 60° C. for 16 hours. The crude rasagiline obtained by this process is very impure and has to be purified by column chromatography.

Another process for the preparation of rasagiline, disclosed in WO 95/11016 and U.S. Pat. No. 5,532,415, is very similar to the process disclosed in U.S. Pat. No. 5,457,133, with the exception that propargyl benzenesulfonate is used instead of propargyl chloride. One of the disclosed examples describes a process, which involves reaction of racemic 1-aminoindan with propargyl benzenesulfonate in the presence of 15% aqueous NaOH in toluene to form racemic rasagiline, which is then resolved with tartaric acid to form the L-tartrate diastereomeric salt of rasagiline by a crystallisation technique. The L-tartrate diastereomeric salt of rasagiline is then reacted with methanesulfonic acid in isopropanol to afford rasagiline mesylate, which is precipitated and dried. However, the inventors have found that the process reported in WO 95/11016 and U.S. Pat. No. 5,532,415 is low yielding, not reproducible and affords a non-crystalline and impure product in terms of chemical purity and polymorphic form. The inventors have also found that the prior art process to prepare rasagiline mesylate affords a product with significant levels of two impurities which have been characterized as dipropargyl indanamine and indanamine mesylate. The prior art does not disclose satisfactory methods for removing the dipropargyl indanamine and indanamine mesylate impurities.

### OBJECT OF THE INVENTION

It is therefore an object of the present invention to provide stable, reproducibly crystalline and pure forms of rasagiline mesylate and methods for preparing such forms.

### SUMMARY OF THE INVENTION

For the purposes of the present invention, rasagiline mesylate is “substantially free” of chemical, dipropargyl indanamine or indanamine mesylate impurities, if it comprises less than 3% of the given impurity, preferably less than 2%, preferably less than 1%, preferably less than 0.5%, preferably less than 0.1%, for example as measured by HPLC.

As used herein, the terms “mixing”, “mixture” and the like may relate to any combination of substances, for example, a solution, a partial solution where the solute is not fully dissolved, a solution of two or more miscible liquids, a slurry and a suspension of any type may all be included.

For the purposes of the present invention, one “volume” or “vol” in relation to a liquid such as a solvent refers to 1 ml of the liquid for each gram of rasagiline mesylate or rasagiline used in the process.

According to a first aspect of the present invention there is provided a crystalline form of tasagiline mesylate characterized by an X-ray diffraction pattern having peaks at 2θ values 9.3, 13.7, 16.4, 16.8 and 18.3±0.2° 2θ.

According to a second aspect of the present invention there is provided a crystalline form of rasagiline mesylate characterized by an X-ray diffraction pattern having peaks at 2θ values 9.3, 13.7, 16.4, 16.8, 18.3, 21.3, 21.7, 22.3, 22.9, 23.1, 24.1, 24.5, 25.3, 26.7 and 27.5±0.2° 2θ.

According to a third aspect of the present invention there is provided a crystalline form of rasagiline mesylate characterized by an X-ray diffraction pattern substantially as shown in FIG. 1.

Preferably the crystalline form according to the first, second or third aspect of the present invention is further characterized by a DSC with an endothermic peak at about 156° C.

Preferably the crystalline form according to the first, second or third aspect of the present invention is substantially free of dipropargyl indanamine impurity and/or substantially free of indanamine mesylate impurity and/or substantially free of chemical impurities.

Preferably the crystalline form according to the first, second or third aspect of the present invention is stable over time, such as over 3, 6 or 12 months.

According to a fourth aspect of the present invention there is provided rasagiline mesylate substantially free of chemical impurities.

According to a fifth aspect of the present invention there is provided rasagiline mesylate substantially free of dipropargyl indanamine impurity.

According to a sixth aspect of the present invention there is provided rasagiline mesylate substantially free of indanamine mesylate impurity.

Preferably the rasagiline mesylate according to the fourth, fifth or sixth aspect of the present invention is crystalline. Preferably the rasagiline mesylate is characterized by an X-ray diffraction pattern having peaks at 20 values 9.3, 13.7, 16.4, 16.8 and 18.3±0.2° 2θ. Preferably the rasagiline mesylate is characterized by an X-ray diffraction pattern having peaks at 2θ values 9.3, 13.7, 16.4, 16.8, 18.3, 21.3, 21.7, 22.3, 22.9, 23.1, 24.1, 24.5, 25.3, 26.7 and 27.5±0.2° 2θ. Preferably the rasagiline mesylate is characterized by an X-ray diffraction pattern substantially as shown in FIG. 1.

Preferably the rasagiline mesylate according to the fourth, fifth or sixth aspect of the present invention is characterized by a DSC with an endothermic peak at about 156° C.

Preferably the rasagiline mesylate according to the fourth, fifth or sixth aspect of the present invention is stable over time, such as over 3, 6 or 12 months.

According to a seventh aspect of the present invention there is provided a process for the preparation of a crystalline or pure form of rasagiline mesylate, comprising the steps of: (a) mixing rasagiline mesylate or rasagiline and methanesulfonic acid in a solvent, and (b) isolating the rasagiline mesylate. Preferably in step (a) rasagiline mesylate is mixed in a solvent.

Preferably the rasagiline mesylate, or the rasagiline and the methanesulfonic acid, is/are dissolved in the solvent to form a solution. Alternatively, the rasagiline mesylate, or the rasagiline and the methanesulfonic acid, is/are mixed in the solvent to form a slurry. Preferably the solution or slurry is heated, preferably to about 40-120° C., preferably for about 10 minutes to 2 hours, preferably for about 10-50 minutes, preferably for about 20-30 minutes. Preferably the solution or slurry is cooled to isolate the rasagiline mesylate, preferably to about 0-25° C., preferably for about 10 minutes to 2 hours, preferably for about 10-50 minutes, preferably for about 30 minutes.

Preferably the solvent used is chosen from an organic solvent or water or a mixture thereof. The organic solvent is preferably a nitrile, an ether, an alcohol, a ketone, a hydrocarbon or an ester, preferably comprising 1 to 12 carbon atoms, more preferably comprising 1 to 6 carbon atoms. The organic solvent is preferably an alcohol, preferably comprising 1 to 12 carbon atoms, more preferably comprising 1 to 6 carbon atoms. In one embodiment the organic solvent is an alcohol other than isopropyl alcohol. More preferably the solvent is water or an organic solvent selected from acetonitrile, THF, methanol, 1-butanol, ethanol, acetone, isopropyl alcohol, tert-butyl methyl ether, cyclohexane, cyclopentyl methyl ether, 2-butanol, 2-butanone, ethyl acetate or isopropyl acetate or a mixture thereof. Most preferably the organic solvent is 1-butanol.

Where the solvent is a nitrile or an alcohol, preferably 1-10 volumes of solvent are used, and preferably the mixture of rasagiline mesylate, or rasagiline and methanesulfonic acid, in the solvent is heated to about 40-60° C. Where the solvent is an ether, a ketone, a hydrocarbon or an ester, preferably 20-40 volumes of solvent are used, and preferably the mixture of rasagiline mesylate, or rasagiline and methanesulfonic acid, in the solvent is heated to about 50-120° C. and/or to about reflux temperature. Where the solvent is water, preferably 1-5 volumes of water are used, and preferably the mixture of rasagiline mesylate, or rasagiline and methanesulfonic acid, in water is heated to or maintained at about 20-30° C.

Preferably the isolated crystalline rasagiline mesylate is dried, preferably at 20-40° C., preferably under vacuum, preferably for 0.5-10 hours, preferably until a constant weight is achieved.

Preferably the rasagiline mesylate according to any one of the first to sixth aspects of the present invention or when prepared by a process according to the seventh aspect of the present invention is suitable for use in medicine, preferably for treating or preventing Parkinson's Disease (both as monotherapy and as adjunct therapy to levodopa), dementia, Alzheimer's Disease, depression, hyperactive syndrome, stroke, brain ischemia, neurotrauma, schizophrenia or multiple sclerosis, most preferably for treating or preventing Parkinson's Disease (both as monotherapy and as adjunct therapy to levodopa).

According to an eighth aspect of the present invention there is provided a pharmaceutical composition comprising the rasagiline mesylate according to any one of the first to sixth aspects of the present invention or when prepared by a process according to the seventh aspect of the present invention. Preferably the pharmaceutical composition is a solid dosage form preferably for oral administration, more preferably a tablet. Preferably the pharmaceutical composition is suitable for the treatment or prevention of Parkinson's Disease (both as monotherapy and as adjunct therapy to levodopa), dementia, Alzheimer's Disease, depression, hyperactive syndrome, stroke, brain ischemia, neurotrauma, schizophrenia or multiple sclerosis. Preferably the pharmaceutical composition is suitable for the treatment or prevention of Parkinson's Disease (both as monotherapy and as adjunct therapy to levodopa).

According to a ninth aspect of the present invention there is provided a use of rasagiline mesylate according to any one of the first to sixth aspects of the present invention or when prepared by a process according to the seventh aspect of the present invention, or a use of the pharmaceutical composition according to the eighth aspect of the present invention, for the preparation of a medicament for the treatment or prevention of Parkinson's Disease (both as monotherapy and as adjunct therapy to levodopa), dementia, Alzheimer's Disease, depression, hyperactive syndrome, stroke, brain ischemia, neurotrauma, schizophrenia or multiple sclerosis. Preferably the medicament is for the treatment or prevention of Parkinson's Disease (both as monotherapy and as adjunct therapy to levodopa).

According to a tenth aspect of the present invention there is provided a method of treating or preventing Parkinson's Disease (both as monotherapy and as adjunct therapy to levodopa), dementia, Alzheimer's Disease, depression, hyperactive syndrome, stroke, brain ischemia, neurotrauma, schizophrenia or multiple sclerosis, the method comprising administering to a patient in need thereof a therapeutically or prophylactically effective amount of rasagiline mesylate according to any one of the first to sixth aspects of the present invention or when prepared by a process according to the seventh aspect of the present invention, or of the pharmaceutical composition according to the eighth aspect of the present invention. Preferably the method is for treating or preventing Parkinson's Disease (both as monotherapy and as adjunct therapy to levodopa). Preferably the patient is a mammal, preferably a human.

### BRIEF DESCRIPTION OF THE ACCOMPANYING FIGURES

FIG. 1 describes the XRPD of the rasagiline mesylate of the present invention.

FIG. 2 describes the DSC of the rasagiline mesylate of the present invention.

### DETAILED DESCRIPTION OF THE INVENTION

The crystalline form in accordance with the invention can be used to advantage in the preparation of pharmaceutical dosage or drug forms. When in particulate form, the crystalline form in accordance with the present invention is stable and free flowing and does not present any of the stability (e.g. polymorphic conversion or chemical conversion) or handling difficulties associated with the prior art form. The novel crystalline form according to the invention, therefore, can be employed in the manufacture of pharmaceutical compositions that do not suffer from the problems, such as inconsistent drug substance dissolution rates and the like, that can be manifest in dosage forms manufactured using previously available forms of rasagiline mesylate.

Accordingly, in further aspects, the present invention provides a method of preparing a pharmaceutical dosage form that utilises the crystalline form in accordance with the present invention. It also provides a pharmaceutical dosage form prepared or preparable by such a method. The dosage form can be a solution or suspension form, but is preferably solid and comprises one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a gelatine material and can include a conventionally prepared granulate of excipients and crystalline form in accordance with the invention.

The crystalline form in accordance with the present invention may also be useful as precursor to other novel or known polymorphic forms of rasagiline mesylate that may be useful in the preparation of pharmaceutical products.

In the processes according to the present invention, the mixture of rasagiline mesylate and solvent is preferably heated and cooled before isolation of the crystalline rasagiline mesylate. Preferably the mixture is heated at 40 to 110° C., preferably 50 to 90° C., more preferably 50 to 80° C., and most preferably 50 to 65° C.

The process disclosed in this application is capable of providing the crystalline form in consistent polymorphic purity and chemical purity irrespective of the scale of preparation.

The process disclosed in this application is capable of providing rasagiline mesylate in very high purity and substantially free of all impurities. A particularly preferred solvent for the preparation of this highly pure rasagiline mesylate is 1-butanol which affords a product with chemical purity greater than 99.9% by HPLC. Therefore a preferred process for the preparation of rasagiline mesylate substantially free of unreacted (R)-1-indanamine mesylate comprises the steps of dissolving rasagiline mesylate in 1-butanol, preferably at around 40 to 50° C., cooling the solution and isolating rasagiline mesylate from the mixture.

In the processes according to the present invention, the rasagiline mesylate is preferably formed by direct reaction of rasagiline base with methanesulfonic acid and subsequent crystallisation (and optionally recrystallisation) of the mesylate salt formed. This is an improved process and more convenient than the prior art process, which affords a less pure product as the mesylate salt is prepared from the corresponding tartrate salt.

A further aspect of the invention also comprises a pharmaceutical composition comprising the crystalline form of the present invention and a pharmaceutically acceptable excipient. Another aspect of the present invention is the pharmaceutical composition containing the crystalline form and uses of the pharmaceutical composition to provide methods of treating patients suffering from Parkinson's Disease, dementia, Alzheimer's Disease, depression, hyperactive syndrome, stroke, brain ischemia, neurotrauma, schizophrenia and multiple sclerosis.

The details of the invention, its objects and advantages are explained hereunder in greater detail in relation to non-limiting examples.

### EXAMPLES

Crude rasagiline mesylate was prepared according to the following procedure and this crude material was recrystallised according to the methods of examples 1-15 to afford the crystalline form according to the present invention.

(R)-1-Indanamine (1 eq) was taken in dry THF (3 vol) and to the cooled reaction mixture, DBU (1.5 eq) was added dropwise. The reaction mixture was stirred for 1 hour at 0-5° C. Propargyl tosylate (1.5 eq) was added dropwise at 0-5° C. and the mixture stirred for 2 hours at 20-25° C. After completion of the reaction, the THF was removed under vacuum and water (3 vol) was added followed by 10% aqueous NaOH (10 vol) and the mixture was stirred for 15 minutes. Extraction was done with DCM, followed by washing with 10% aqueous NaOH and water. The DCM was removed under vacuum at 40° C. to afford rasagiline base.

The rasagiline base (1 eq) prepared above was taken in isopropyl alcohol (3 vol). The mixture was cooled to 0-5° C. To this was added, dropwise, a solution of methanesulfonic acid (1 eq) in isopropyl alcohol (2 vol) at 0-5° C. and stirred for 1 hour. A white solid crystallised which was filtered and washed with isopropyl alcohol (1 vol). The crystalline rasagiline mesylate formed was dried under vacuum at 40° C. for 3 hours.

### Example 1

Rasagiline mesylate (1 eq) was taken in acetonitrile (4 vol) and heated to 55° C. for 20-30 minutes until a clear solution was obtained The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 2

Rasagiline mesylate (1 eq) was taken in THF (20 vol) and heated to 66° C. for 20-30 minutes. A slurry was obtained. The slurry was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 3

Rasagiline mesylate (1 eq) was taken in 1-butanol (3 vol) and heated to 55° C. for 20-30 minutes until a clear solution was obtained The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 4

Rasagiline mesylate (1 eq) was taken in 1-butanol (3 vol) and heated to 45° C. for 20-30 minutes until a clear solution was obtained. The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 5

Rasagiline mesylate (1 eq) was taken in ethanol (3 vol) and heated to 40° C. for 20-30 minutes until a clear solution was obtained The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 6

Rasagiline mesylate (1 eq) was taken in acetone (40 vol) and heated to 55° C. for 20-30 minutes until a clear solution was obtained The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 7

Rasagiline mesylate (1 eq) was taken in isopropyl acetate (40 vol) and heated to 85° C. for 20-30 minutes. A slurry was obtained. The slurry was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 8

Rasagiline mesylate (1 eq) was taken in water (1 vol) at 25° C. for 20-30 minutes. A clear solution was obtained. The solution was cooled to 0-5° C., maintained for 30 minutes and the precipitated solid filtered. The solid product was dried at 25-30° C. under vacuum for 2 hours. XRPD and DSC analysis data confirmed that the product obtained was a crystalline form of rasagiline mesylate.

### Example 9

Rasagiline mesylate (1 eq) was taken in tert-butyl methyl ether (40 vol) and heated to 55° C. for 20-30 minutes. A slurry was obtained The slurry was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 10

Rasagiline mesylate (1 eq) was taken in cyclohexane (40 vol) and heated to 80° C. for 20-30 minutes. A slurry was obtained The slurry was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 11

Rasagiline mesylate (1 eq) was taken in cyclopentyl methyl ether (40 vol) and heated to 105° C. for 20-30 minutes until a clear solution was obtained. The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 12

Rasagiline mesylate (1 eq) was taken in 2-butanol (3 vol) and heated to 58° C. for 20-30 minutes until a clear solution was obtained. The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 13

Rasagiline mesylate (1 eq) was taken in 2-butanone (30 vol) and heated to 80° C. for 20-30 minutes until a clear solution was obtained. The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 14

Rasagiline mesylate (1 eq) was taken in isopropyl alcohol (5 vol) and heated to 55° C. for 20-30 minutes until a clear solution was obtained. The solution was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

### Example 15

Rasagiline mesylate (1 eq) was taken in ethyl acetate (25 vol) and heated to 77° C. for 20-30 minutes. A slurry was obtained. The slurry was cooled to 25° C. for 30 minutes and filtered. The solid product was dried at 25-30° C. under vacuum for 2 hours. XRPD and DSC analysis data confirmed that the product obtained was a crystalline form of rasagiline mesylate.

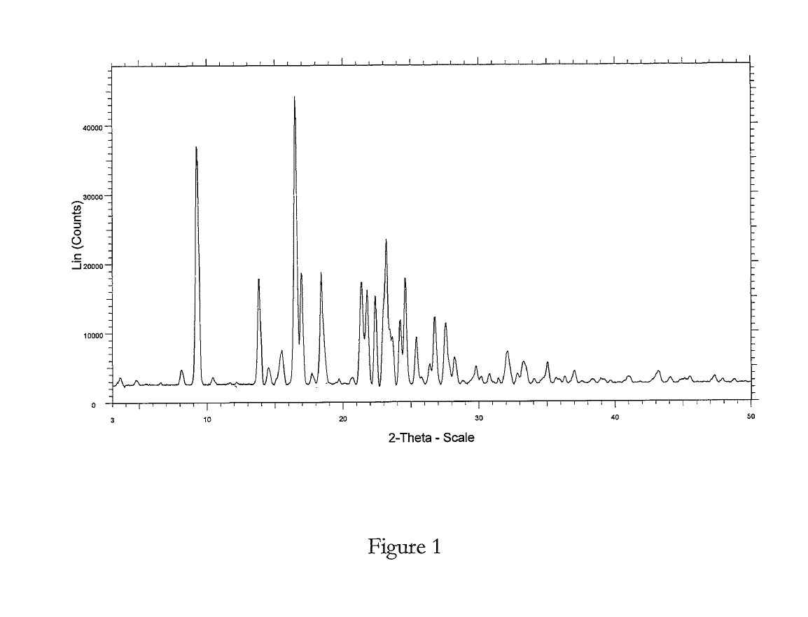

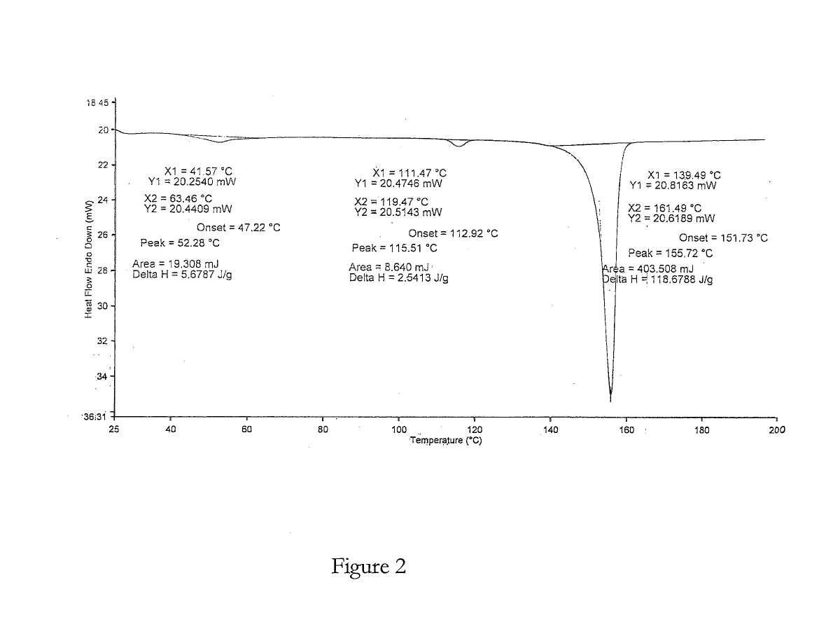

The rasagiline mesylate products obtained in examples 1-15 exhibited the XRPD shown in FIG. 1 and the DSC shown in FIG. 2. The XRPD spectra were recorded on a Bruker D8 Advance Instrument, using Cu radiation as the X-ray source and lynxeye as the detector, with a 20 range of from 3 to 50°.

The rasagiline mesylate products obtained in examples 1-15 were found to have a chemical purity of >99.0% as measured by HPLC.

The rasagiline mesylate products obtained in examples 1-15 were subjected to 6 months stability studies and found to be highly stable.

It will be understood that the present invention has been described above by way of example only. The examples are not intended to limit the scope of the invention. Various modifications and embodiments can be made without departing from the scope and spirit of the invention, which is defined by the following claims only.

1-30. (canceled)

31. A crystalline form of rasagiline mesylate characterized by an X-ray diffraction pattern having peaks at 2θ values 9.3, 13.7, 16.4, 16.8 and 18.3±0.2° 2θ.

32. A crystalline form of rasagiline mesylate according to claim 31, characterized by:
(i) an X-ray diffraction pattern having peaks at 2θ values 9.3, 13.7, 16.4, 16.8, 18.3, 21.3, 21.7, 22.3, 22.9, 23.1, 24.1, 24.5, 25.3, 26.7 and 27.5±0.2° 2θ; and/or
(ii) an X-ray diffraction pattern substantially as shown in FIG. 1; and/or
(iii) a DSC with an endothermic peak at about 156° C.

33. Rasagiline mesylate substantially free of chemical impurities.

34. Rasagiline mesylate substantially free of dipropargyl indanamine.

35. Rasagiline mesylate substantially free of indanamine mesylate.

36. A process for the preparation of a crystalline or pure form of rasagiline mesylate, comprising the steps of: (a) mixing rasagiline mesylate or rasagiline and methanesulfonic acid in a solvent, and (b) isolating the rasagiline mesylate.

37. A process according to claim 36, wherein in step (a) rasagiline mesylate is mixed in a solvent.

38. A process according to claim 36, wherein the rasagiline mesylate, or the rasagiline and the methanesulfonic acid, is/are:
(i) dissolved in the solvent to form a solution and the solution is cooled to isolate the rasagiline mesylate; or
(ii) mixed in the solvent to form a slurry and the slurry is cooled to isolate the rasagiline mesylate.

39. A process according to claim 36, wherein the solvent is:
(i) chosen from an organic solvent or water or a mixture thereof; and/or
(ii) water; and/or
(iii) an organic solvent; and/or
(iv) an organic solvent selected from acetonitrile, THF, methanol, 1-butanol, ethanol, acetone, isopropyl alcohol, tert-butyl methyl ether, cyclohexane, cyclopentyl methyl ether, 2-butanol, 2-butanone, ethyl acetate or isopropyl acetate or a mixture thereof.

40. A pharmaceutical composition comprising the rasagiline mesylate according to claim 31.

41. A pharmaceutical composition comprising the rasagiline mesylate according to claim 33.

42. A pharmaceutical composition comprising the rasagiline mesylate according to claim 34.

43. A pharmaceutical composition comprising the rasagiline mesylate according to claim 35.

44. A pharmaceutical composition comprising the rasagiline mesylate when prepared by a process according to claim 36.

45. The pharmaceutical composition according to claim 40, which is:
(i) a solid dosage form for oral administration; and/or
(ii) a tablet for oral administration.

46. The pharmaceutical composition according to claim 41, which is:
(i) a solid dosage form for oral administration; and/or
(ii) a tablet for oral administration.

47. The pharmaceutical composition according to claim 42, which is:
(i) a solid dosage form for oral administration; and/or
(ii) a tablet for oral administration.

48. The pharmaceutical composition according to claim 43, which is:
(i) a solid dosage form for oral administration; and/or
(ii) a tablet for oral administration.

49. The pharmaceutical composition according to claim 44, which is:
(i) a solid dosage form for oral administration; and/or
(ii) a tablet for oral administration.

50. A method of treating or preventing Parkinson's Disease, including as monotherapy or as adjunct therapy to levodopa, dementia, Alzheimer's Disease, depression, hyperactive syndrome, stroke, brain ischemia, neurotrauma, schizophrenia or multiple sclerosis, the method comprising administering to a patient in need thereof a therapeutically or prophylactically effective amount of the rasagiline mesylate according to claim 31.

51. A method of treating or preventing Parkinson's Disease, including as monotherapy or as adjunct therapy to levodopa, dementia, Alzheimer's Disease, depression, hyperactive syndrome, stroke, brain ischemia, neurotrauma, schizophrenia or multiple sclerosis, the method comprising administering to a patient in need thereof a therapeutically or prophylactically effective amount of the rasagiline mesylate according to claim 33.

52. A method of treating or preventing Parkinson's Disease, including as monotherapy or as adjunct therapy to levodopa, dementia, Alzheimer's Disease, depression, hyperactive syndrome, stroke, brain ischemia, neurotrauma, schizophrenia or multiple sclerosis, the method comprising administering to a patient in need thereof a therapeutically or prophylactically effective amount of the rasagiline mesylate according to claim 34.

53. A method of treating or preventing Parkinson's Disease, including as monotherapy or as adjunct therapy to levodopa, dementia, Alzheimer's Disease, depression, hyperactive syndrome, stroke, brain ischemia, neurotrauma, schizophrenia or multiple sclerosis, the method comprising administering to a patient in need thereof a therapeutically or prophylactically effective amount of the rasagiline mesylate according to claim 35.

54. A method of treating or preventing Parkinson's Disease, including as monotherapy or as adjunct therapy to levodopa, dementia, Alzheimer's Disease, depression, hyperactive syndrome, stroke, brain ischemia, neurotrauma, schizophrenia or multiple sclerosis, the method comprising administering to a patient in need thereof a therapeutically or prophylactically effective amount of the rasagiline mesylate when prepared by a process according to claim 36.

55. A method according to claim 50:
(i) for treating or preventing Parkinson's Disease, including as monotherapy or as adjunct therapy to levodopa; and/or
(ii) wherein the patient is a mammal; and/or
(iii) wherein the patient is a human.

56. A method according to claim 51:
(i) for treating or preventing Parkinson's Disease, including as monotherapy or as adjunct therapy to levodopa; and/or
(ii) wherein the patient is a mammal; and/or
(iii) wherein the patient is a human.

57. A method according to claim 52:
(i) for treating or preventing Parkinson's Disease, including as monotherapy or as adjunct therapy to levodopa; and/or
(ii) wherein the patient is a mammal; and/or
(iii) wherein the patient is a human.

58. A method according to claim 53:
(i) for treating or preventing Parkinson's Disease, including as monotherapy or as adjunct therapy to levodopa; and/or
(ii) wherein the patient is a mammal; and/or
(iii) wherein the patient is a human.

59. A method according to claim 54:
(i) for treating or preventing Parkinson's Disease, including as monotherapy or as adjunct therapy to levodopa; and/or
(ii) wherein the patient is a mammal; and/or
(iii) wherein the patient is a human.
